(19) United States
(12) Patent Application Publication
(10) Pub. No.: US 2015/0164605 A1
(43) Pub. Date: Jun. 18, 2015

(54) METHODS AND SYSTEMS FOR INTERVENTIONAL IMAGING

(71) Applicant: General Electric Company, Schenectady, NY (US)

(72) Inventors: Kedar Anil Patwardhan, Latham, NY (US); James Vradenburg Miller, Schenectady, NY (US); Tai-Peng Tian, Niskayuna, NY (US)

(73) Assignee: General Electric Company, Schenectady, NY (US)

(21) Appl. No.: 14/106,091

(22) Filed: Dec. 13, 2013

(51) Int. Cl. A61B 19/00; A61B 5/00; A61B 8/00; A61B 6/03; A61B 1/04; A61B 17/00; G01R 33/28; A61B 8/08

(52) U.S. Cl. CPC A61B 19/5244; G01R 33/285; A61B 5/0035; A61B 8/0841; A61B 6/032; A61B 6/037; A61B 5/0066; A61B 1/04; A61B 17/00234; A61B 8/085; A61B 5/4887; A61B 8/466; A61B 2019/5251; A61B 2019/5255; A61B 2019/5276; A61B 2019/524; A61B 2019/5236; A61B 2019/5263; A61B 5/055

(57) ABSTRACT

Methods and systems for imaging a subject are presented. A series of volumetric images corresponding to a volume of interest in the subject is received during an interventional procedure. One or more of anatomical structures in at least one volumetric image selected from the series of volumetric images are detected. Detecting the anatomical structures includes determining an originally acquired view of the anatomical structures in the selected volumetric image. An optimal view of the anatomical structures is determined for performing a desired imaging task during the interventional procedure. The detected anatomical structures are automatically reoriented to transform the originally acquired view of the detected anatomical structures into a reoriented view. One or more obstructing structures are automatically removed from the reoriented view to generate the optimal view of the detected anatomical structures. The selected volumetric image including the optimal view of the detected anatomical structures is displayed in real-time.

### BACKGROUND

Embodiments of the present disclosure relate generally to interventional imaging and, more particularly, to methods and systems for optimal visualization of a target region for use in interventional procedures.

Interventional techniques are widely used for managing a plurality of life-threatening medical conditions. Particularly, certain interventional techniques entail minimally invasive image-guided procedures that provide a cost-effective alternative to invasive surgery. Additionally, the minimally invasive interventional procedures minimize pain and trauma caused to a patient, thereby resulting in shorter hospital stays. Accordingly, minimally invasive transcatheter therapies have found extensive use, for example, in diagnosis and treatment of valvular and congenital heart diseases. The transcatheter therapies may be further facilitated through multi-modality imaging that aids in planning, guidance, and evaluation of procedure related outcomes and complications.

By way of example, interventional procedures such as transesophageal echocardiography (TEE) and/or intracardiac echocardiography (ICE) may be used to provide high resolution images of intracardiac anatomy. The high resolution images, in turn, allow for real-time guidance of interventional devices during structural heart disease (SHD) interventions such as transcatheter aortic valve implantation (TAVI), paravalvular regurgitation repair, and/or mitral valve interventions.

Particularly, TEE may be used to diagnose and/or treat SHD and/or electrophysiological disorders such as arrhythmias. To that end, TEE employs a probe positioned inside the esophagus of a patient to visualize cardiac structures. Although TEE allows for well-defined workflows and good image quality, TEE may not be suitable for all cardiac interventions. For example, TEE may provide only limited visualization of certain anterior cardiac features due to imaging artifacts caused due to shadowing from surrounding structures and/or a lack of far-field exposure. Further, manipulating the TEE probe may require a specialist echo-cardiographer. Additionally, TEE may be employed only for short procedures to prevent any esophageal trauma in patients.

Accordingly, in certain longer interventional procedures, ICE may be used to provide high resolution images of cardiac structures, often under conscious sedation of the patient. Furthermore, ICE equipment may be interfaced with other interventional imaging systems, thus allowing for supplemental imaging that may provide additional information for device guidance, diagnosis, and/or treatment. For example, a CT imaging system may be used to provide supplemental views of an anatomy of interest in real-time to facilitate ICE-assisted interventional procedures.

Typically, during the ICE-assisted interventional procedures, an ICE catheter may be inserted into a vein, such as the femoral vein, to image a cardiac region of interest (ROI). Particularly, the ICE catheter may include an imager configured to generate volumetric images of the cardiac ROI corresponding to the interventional procedure being performed. The ICE images, thus generated, may be used to provide a medical practitioner with real-time guidance for positioning and/or navigating an interventional device such as a stent, an ablation catheter, or a needle within the patient's body. For example, the ICE images may be used to provide the medical practitioner with an illustrative map to navigate the ablation catheter within the patient's body to provide therapy to desired regions of interest (ROIs). Additionally, the images may be used, for example, to obtain basic cardiac measurements, visualize valve structure, and measure septal defect dimensions to aid the medical practitioner in accurately diagnosing a medical condition of the patient.

However, maneuvering and/or orienting the ICE catheter within open cavities of the heart to acquire a desired view of the cardiac ROI relevant to a current patient exam may be difficult. Specifically, a native visualization on the imager may assume an originally acquired view direction, which may not be sufficient to provide a clinically useful view of the desired ROI. Accordingly, in conventional ICE systems, the medical practitioner may manually configure one or more controls corresponding to the ICE system to orient the image to provide a better viewing direction. Additionally, the medical practitioner may also manually configure the ICE system controls to define clipping planes to visualize desired ROIs, while removing clutter from a selected field-of-view (FOV).

However, manual configuration of the system controls to refine the FOV to acquire a desired image of a cardiac ROI may be a complicated and time consuming procedure. Furthermore, manual configuration of the system controls may interrupt the interventional procedure, thus prolonging duration of the procedure. The prolonged procedure time, in turn, may increase a risk of trauma to the cardiac tissues. Furthermore, the prolonged procedure time may also impede real-time diagnosis and/or guidance of an interventional device.

### BRIEF DESCRIPTION

In accordance with an aspect of the present disclosure, a method for imaging a subject is disclosed. The method includes receiving a series of volumetric images corresponding to a volume of interest in the subject during an interventional procedure. Further, the method includes detecting one or more anatomical structures in at least one volumetric image selected from the series of volumetric images, where detecting the anatomical structures includes determining an originally acquired view of the anatomical structures in the selected volumetric image. Additionally, the method includes determining an optimal view of the one or more anatomical structures of interest for performing a desired imaging task during the interventional procedure. Moreover, the method includes automatically reorienting the detected anatomical structures in the selected volumetric image to transform the originally acquired view of the detected anatomical structures into a reoriented view. Furthermore, the method includes automatically removing one or more obstructing structures from the reoriented view in the selected volumetric image to generate the optimal view of the detected anatomical structures. Additionally, the method includes displaying the selected volumetric image comprising the optimal view of the detected anatomical structures in real-time.

In accordance with another aspect of the present disclosure, an imaging system is presented. The system includes an acquisition subsystem configured to acquire a series of volumetric images corresponding to a volume of interest in a subject. Further, the system includes a processing unit communicatively coupled to the acquisition subsystem and configured to detect one or more anatomical structures in at least one volumetric image selected from the series of volumetric images, where detecting the anatomical structures includes determining an originally acquired view of the anatomical structures in the selected volumetric image. Moreover, the processing unit is configured to determine an optimal view of the one or more anatomical structures of interest for performing a desired imaging task during the interventional procedure. Additionally, the processing unit is configured to automatically reorient the detected anatomical structures in the selected volumetric image to transform the originally acquired view of the detected anatomical structures into a reoriented view. Furthermore, the processing unit is configured to automatically remove one or more obstructing structures from the reoriented view in the selected volumetric image to generate the optimal view of the detected anatomical structures. Moreover, the system also includes a display operatively coupled to at least the processing unit and configured to display the selected volumetric image comprising the optimal view of the detected anatomical structures in real-time.

In accordance with a further aspect of the present disclosure, non-transitory computer readable medium that stores instructions executable by one or more processors to perform a method for imaging a subject is presented. The method includes receiving a series of volumetric images corresponding to a volume of interest in the subject during an interventional procedure. Further, the method includes detecting one or more anatomical structures in at least one volumetric image selected from the series of volumetric images, where detecting the anatomical structures includes determining an originally acquired view of the anatomical structures in the selected volumetric image. Additionally, the method includes determining an optimal view of the one or more anatomical structures of interest for performing a desired imaging task during the interventional procedure. Moreover, the method includes automatically reorienting the detected anatomical structures in the selected volumetric image to transform the originally acquired view of the detected anatomical structures into a reoriented view. Furthermore, the method includes automatically removing one or more obstructing structures from the reoriented view in the selected volumetric image to generate the optimal view of the detected anatomical structures. Additionally, the method includes displaying the selected volumetric image comprising the optimal view of the detected anatomical structures in real-time.

### DRAWINGS

These and other features, aspects, and advantages of the present disclosure will become better understood when the following detailed description is read with reference to the accompanying drawings in which like characters represent like parts throughout the drawings, wherein:

FIG. 1 is a schematic representation of an exemplary imaging system, in accordance with aspects of the present disclosure;

FIG. 2 is a flow diagram illustrating an exemplary method for interventional imaging, in accordance with aspects of the present disclosure;

FIG. 3 is a diagrammatical representation of a default visualization of a volume of interest (VOI) corresponding to a subject using a computed tomography (CT) system, in accordance with aspects of the present disclosure;

FIG. 4 is a diagrammatical representation of a reoriented and/or repositioned view of the VOI of FIG. 3 generated using the method of FIG. 2, in accordance with aspects of the present disclosure;

FIG. 5 is an exemplary image depicting a default side view of a cardiac valve acquired by a TEE probe;

FIG. 6 is an exemplary image depicting a default axial view of the cardiac valve of FIG. 5 acquired by the TEE probe;

FIG. 7 is an exemplary image depicting an optimal view of the cardiac valve of FIG. 5 generated using the method of FIG. 2 when the valve is closed; and

FIG. 8 is an exemplary image depicting an optimal view of the cardiac valve of FIG. 5 generated using the method of FIG. 2 when the valve is open.

### DETAILED DESCRIPTION

The following description presents systems and methods for optimal visualization of target anatomical structures of interest for use during interventional procedures. Particularly, certain embodiments illustrated herein describe methods and systems that are configured to automatically process a series of volumetric images to transform an originally acquired view of a target structure into a desired view that is relevant to an interventional procedure being performed. For example, a technical effect of the present disclosure is to provide automatic reorientation of the originally acquired view of the target structure such as a pulmonary vein in the cardiac region of a patient to provide a reoriented view of the target structure. Furthermore, one or more obstructing structures such as a septum may be removed from the reoriented view to provide an optimal view for ablating desired regions of the pulmonary vein. Automatic reorientation and/or removal of obstructing anatomy preclude need for time consuming manual configuration of system controls, thereby expediting the interventional procedure.

Accordingly, embodiments of the present systems and methods allow for automatic customization of one or more imaging and/or viewing parameters that may be used to display the optimal view of the target structure. The specific imaging and/or viewing parameters to be customized may be determined based on the interventional procedure being performed. In one example, the imaging parameters may include a desired pulse sequence, a desired spatial location, a depth of acquisition, and/or a desired FOV of the target structure. Further, the viewing parameters may include viewing orientation, clipping planes, image contrast, and/or spatial resolution.

Embodiments of the present systems and methods may use the customized imaging and/or viewing parameters, for example, to allow for automatic reorientation, clipping, and/or contrast enhancement corresponding to the volumetric image. The volumetric image, thus processed, may be visualized on the display to provide a medical practitioner with more definitive information corresponding to the target structure in real-time compared to conventional imaging systems. In one embodiment, this information may be used to provide automated guidance for positioning and/or navigating one or more interventional devices through the body of the patient. Additionally, in certain embodiments, the reorientation and/or obstruction-related information may be used to provide suitable suggestions to a user regarding manipulating an imaging catheter to better capture the target structure in a subsequent scan.

Although embodiments of the present disclosure are described with reference to ICE, use of the present systems and methods in other imaging applications and/or modalities is also contemplated. For example, the present systems and methods may be implemented in Transthoracic echocardiography (TTE) systems, TEE systems, and/or Optical Coherence Tomography (OCT) systems. Embodiments of the present systems and methods may also be used to more accurately diagnose and stage coronary artery disease and to help monitor therapies including, high intensity focused ultrasound (HIFU), radiofrequency ablation (RFA), and brachytherapy by providing an optimal view of the target structure that allows for more accurate structural and functional measurements.

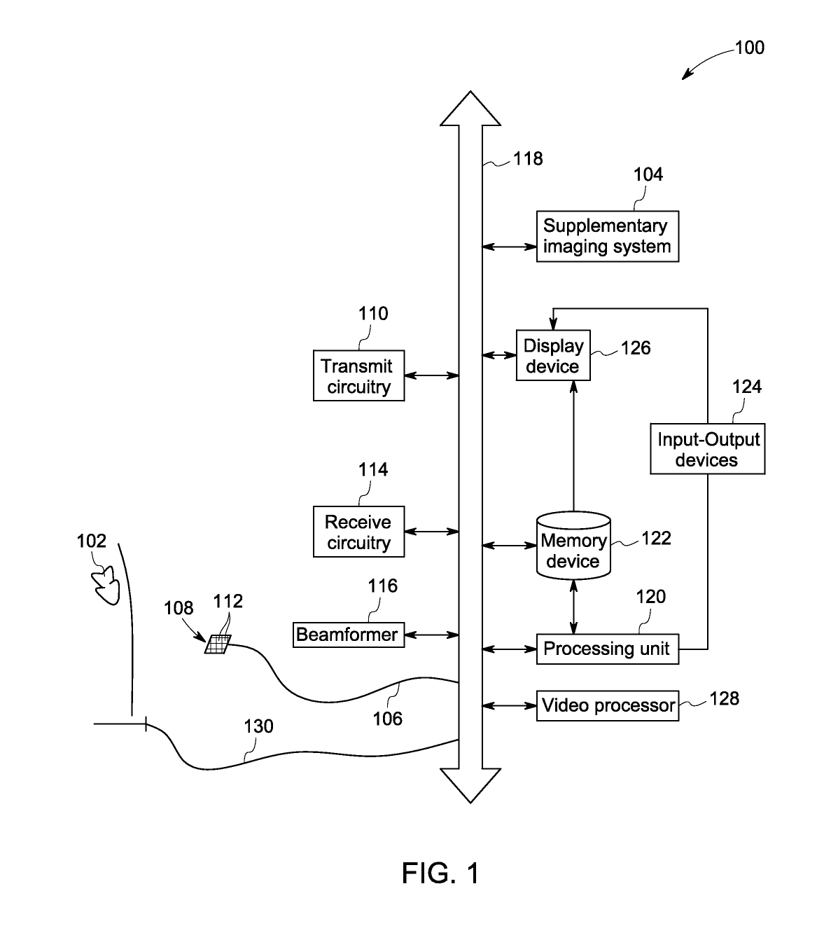

Moreover, at least some of these systems and applications may also be used in non-destructive testing, fluid flow monitoring, and/or other chemical and biological applications. An exemplary environment that is suitable for practicing various implementations of the present system is discussed in the following sections with reference to FIG. 1.

FIG. 1 illustrates an exemplary imaging system 100 for optimal visualization of a target structure 102 for use during interventional procedures. For discussion purposes, the system 100 is described with reference to an ICE system. However, as previously noted, in certain embodiments, the system 100 may be implemented in other interventional imaging systems such as a TTE system, a TEE system, an OCT system, a magnetic resonance imaging (MRI) system, a CT system, a positron emission tomography (PET) system, and/or an X-ray system. Additionally, it may be noted that although the present embodiment is described with reference to imaging a cardiac region corresponding to a patient, certain embodiments of the system 100 may be used with other biological tissues such as lymph vessels, cerebral vessels, and/or in non-biological materials.

In one embodiment, the system 100 employs ultrasound signals to acquire image data corresponding to the target structure 102 in a subject. Moreover, the system 100 may combine the acquired image data corresponding to the target structure 102, for example the cardiac region, with supplementary image data. The supplementary image data, for example, may include previously acquired images and/or real-time intra-operative image data generated by a supplementary imaging system 104 such as a CT, MRI, PET, ultrasound, fluoroscopy, electrophysiology, and/or X-ray system. Specifically, a combination of the acquired image data, and/or supplementary image data may allow for generation of a composite image that provides a greater volume of medical information for use in accurate guidance for an interventional procedure and/or for providing more accurate anatomical measurements.

Accordingly, in one embodiment, the system 100 includes an interventional device such as an endoscope, a laparoscope, a needle, and/or a catheter 106. The catheter 106 is adapted for use in a confined medical or surgical environment such as a body cavity, orifice, or chamber corresponding to a subject. The catheter 106 may further include at least one imaging subsystem 108 disposed at a distal end of the catheter 106. The imaging subsystem 108 may be configured to generate cross-sectional images of the target structure 102 for evaluating one or more corresponding characteristics. Particularly, in one embodiment, imaging subsystem 108 is configured to acquire a series of three-dimensional (3D) and/or four-dimensional (4D) ultrasound images corresponding to the subject. In certain embodiments, the system 100 may be configured to generate the 3D model relative to time, thereby generating a 4D model or image corresponding to the target structure such as the heart of the patient. The system 100 may use the 3D and/or 4D image data, for example, to visualize a 4D model of the target structure 102 for providing a medical practitioner with real-time guidance for navigating the catheter 106 within one or more chambers of the heart.

To that end, in certain embodiments, the imaging subsystem 108 includes transmit circuitry 110 that may be configured to generate a pulsed waveform to drive an array of transducer elements 112. Particularly, the pulsed waveform drives the array of transducer elements 112 to emit ultrasonic pulses into a body or volume of interest in the subject. At least a portion of the ultrasonic pulses generated by the transducer elements 112 back-scatter from the target structure 102 to produce echoes that return to the transducer elements 112 and are received by receive circuitry 114 for further processing.

In one embodiment, the receive circuitry 114 may be operatively coupled to a beamformer 116 that may be configured to process the received echoes and output corresponding radio frequency (RF) signals. Although, FIG. 1 illustrates the transducer elements 112, the transmit circuitry 110, the receive circuitry 114, and the beamformer 116 as distinct elements, in certain embodiments, one or more of these elements may be implemented together as an independent acquisition subsystem in the system 100. The acquisition subsystem may be configured to acquire image data corresponding to the subject, such as a patient, for further processing. As used herein, subject refers to any human or animal subject that may be imaged using the present system.

Further, the system 100 includes a processing unit 120 communicatively coupled to the acquisition subsystem over a communications network 118. The processing unit 120 may be configured to receive and process the acquired image data, for example, the RF signals according to a plurality of selectable ultrasound imaging modes in near real-time and/or offline mode. To that end, the processing unit 120 may be operatively coupled to the beamformer 116, the transducer probe 116, and/or the receive circuitry 114. In one example, the processing unit 120 may include devices such as one or more general-purpose or application-specific processors, digital signal processors, microcomputers, microcontrollers, Application Specific Integrated Circuits (ASICs), Field Programmable Gate Arrays (FPGA), or other suitable devices in communication with other components of the system 100.

In certain embodiments, the processing unit 120 may be configured to provide control and timing signals for selectively configuring one or more imaging and/or viewing parameters for performing a desired imaging task. By way of example, the processing unit 120 may be configured to automatically adjust FOV, spatial resolution, frame rate, depth, and/or frequency of ultrasound signals used for imaging the target structure 102.

Moreover, in one embodiment, the processing unit 120 may be configured to store the acquired volumetric images, the imaging parameters, and/or viewing parameters in a memory device 122. The memory device 122, for example, may include storage devices such as a random access memory, a read only memory, a disc drive, solid-state memory device, and/or a flash memory. Additionally, the processing unit 120 may display the volumetric images and or information derived from the image to a user, such as a cardiologist, for further assessment.

Accordingly, in certain embodiments, the processing unit 120 may be coupled to one or more input-output devices 124 for communicating information and/or receiving commands and inputs from the user. The input-output devices 124, for example, may include devices such as a keyboard, a touchscreen, a microphone, a mouse, a control panel, a display device 126, a foot switch, a hand switch, and/or a button. In one embodiment, the display device 126 may include a graphical user interface (GUI) for providing the user with configurable options for imaging desired regions of the subject. By way of example, the configurable options may include a selectable volumetric image, a selectable ROI, a desired scan plane, a delay profile, a designated pulse sequence, a desired pulse repetition frequency, and/or other suitable system settings used to image the desired ROI. Additionally, the configurable options may include a choice of image-derived information to be communicated to the user. The image-derived information, for example, may include a position and/or orientation of an interventional device, a magnitude of strain, and/or a determined value of stiffness in a target region estimated from the received signals.

In one embodiment, the processing unit 120 may be configured to process the RF signal data to generate the requested image-derived information based on user input. Particularly, the processing unit 120 may be configured to process the RF signal data to generate 2D, 3D, and/or four-dimensional (4D) datasets based on specific scanning and/or user-defined requirements. Additionally, in certain embodiments, the processing unit 120 may be configured to process the RF signal data to generate the volumetric images in real-time while scanning the target region and receiving corresponding echo signals. As used herein, the term “real-time” may be used to refer to an imaging rate upwards of about 30 volumetric images per second with a delay of less than 1 second. Additionally, in one embodiment, the processing unit 120 may be configured to customize the delay in reconstructing and rendering the volumetric images based on specific system-based and/or application-specific requirements. Further, the processing unit 120 may be configured to process the RF signal data such that a resulting image is rendered, for example, at the rate of 30 volumetric images per second on the associated display device 126 that is communicatively coupled to the processing unit 120.

In one embodiment, the display device 126 may be a local device. Alternatively, the display device 126 may be remotely located to allow a remotely located medical practitioner to track the image-derived information corresponding to the subject. In certain embodiments, the processing unit 120 may be configured to update the volumetric images on the display device 126 in an offline and/or delayed update mode. Particularly, the volumetric images may be updated in the offline mode based on the echoes received over a determined period of time. Alternatively, the processing unit 120 may be configured to dynamically update the volumetric images and sequentially display the updated volumetric images on the display device 126 as and when additional volumes of ultrasound data are acquired.

With continued reference, to FIG. 1, in certain embodiments, the system 100 may further include a video processor 128 that may be configured to perform one or more functions of the processing unit 120. For example, the video processor 128 may be configured to digitize the received echoes and output a resulting digital video stream on the display device 126. In one embodiment, the video processor 128 may be configured to display the volumetric images on the display device 126, for example, using a Cartesian coordinate system. Particularly, in certain embodiments, one or more of the system 100, the supplementary imaging system 104, and/or the catheter 106 may be calibrated and/or registered on a common coordinate system to allow for visualization of a change in a FOV of the target structure 102 with a corresponding change in the position and/orientation of the catheter 106. Accordingly, the display device 126 may be used to provide real-time feedback to the medical practitioner regarding a current view corresponding to the target structure 102 and/or an interventional device 130 such as an ablation catheter employed to perform intervention at a site corresponding to the target structure 102.

However, visualizing the structures within the chambers of the heart in a desired FOV determined to be suitable for a patient exam being undertaken may be a challenging procedure. A high degree of freedom corresponding to the imaging subsystem 108 disposed at the distal end of the catheter 106 may complicate maneuvering and/or orienting the ICE catheter 106 within open cavities of the heart. Optimally positioning the imaging subsystem 108 to acquire image data corresponding to the desired FOV of the target structure 102, therefore, may be complicated and may often depend upon a skill and experience of a cardiologist. Even an experienced cardiologist, however, may need to expend a substantial amount of time to manually configure system controls to acquire a clinically acceptable view of the target structure 102. The substantial time taken to manually configure the system controls may interrupt the interventional procedure, while impeding real-time diagnosis and/or guidance of the interventional device 130.

Embodiments of the present system 100, however, allow for automatic processing of acquired volumetric images to visualize the target structure 102 in the desired FOV without employing repeated manual reconfigurations of the system controls. The desired FOV may correspond to an imaging plane that satisfies one or more statutory, clinical, application-specific, and/or user-defined specifications, thereby allowing for real-time tracking of the interventional device 130, accurate measurements of the patient anatomy, and/or efficient evaluation of the target structure 102.

Specifically, in one embodiment, the video processor 128 may be configured to process the acquired volumetric image to automatically reposition and/or reorient the volumetric image to allow for optimal visualization of the target structure 102. To that end, the video processor 128 may be configured to identify one or more anatomical structures of interest from each volumetric image. In one embodiment, the video processor 128 may identify and label the anatomical structures of interest through use of a surgical atlas, a predetermined anatomical model, a supervised machine learning method, patient information gathered from previous medical examinations, and/or other standardized information. In certain embodiments, data from the supplementary imaging system 104 may also be used to aid in identifying the anatomical structures.

Access to the anatomical labeling information corresponding to the patient provides the video processor 128 with comprehensive awareness of the patient anatomy, specifically coordinate locations corresponding to one or more anatomical structures in resulting images. Such comprehensive anatomy awareness provides the system 100 with ample flexibility to automatically customize and render an optimal view of the target structure 102 in real-time.

Additionally, the anatomy awareness may also allow the video processor 128 may automatically remove extraneous data from the volumetric image. The extraneous data, for example, may be determined based on the target structure 102 being imaged and the specific diagnostic and/or interventional information being sought from the generated image. In one embodiment, the extraneous data may be removed from the volumetric image by automatically clipping out, cropping, and/or segmenting the volumetric image.

Additionally, the video processor 128 may rotate and/or reorient the volumetric image such that the anatomical structure such as the pulmonary vein may be positioned and/or oriented on the display device 126 to allow for real-time tracking and/or guidance for movement of the interventional device 130 within one or more cardiac chambers of the heart. A suitable position and/or orientation of the pulmonary vein for use in providing relevant information for real-time tracking and/or guidance may be predetermined based on expert knowledge, user input, and/or historical medical information.

Further, the video processor 128 may analyze an image volume corresponding to structures in the volumetric image other than the pulmonary vein. For example, when imaging the pulmonary vein, the video processor 128 may remove regions in the volumetric image corresponding to the septum and/or echo artifacts caused by the circulating blood to unclutter the volumetric image. Specifically, the video processor 128 may remove the obstructing regions in the volumetric image to render an optimal view that brings a relevant portion of the heart including the pulmonary vein into greater focus.

In certain embodiments, the video processor 128 may be configured to display the optimal view of the target structure 102 along with patient-specific diagnostic and/or therapeutic information in real-time. The video processor 128 may also be configured to supplement the optimal view of the target structure 102 with additional views of the target structure 102 that are acquired by the supplementary imaging system 104. As previously noted, use of the additional views may aid in providing more definitive information corresponding to the target structure 102. Accordingly, in one embodiment, the video processor 128 may be configured to display a composite volumetric image that combines the reoriented and/or repositioned view of the anatomical strictures with the supplementary views to generate the optimal view.

Additionally, in one embodiment, the video processor 128 may be configured to determine and communicate a quality indicator representative of a suitability of each of the originally acquired views of the volumetric images to a user for performing a desired imaging task. In one embodiment, the quality indicator may allow the medical practitioner to ascertain how different the originally acquired view is from the optimal view of the target structure 102. Thus, the quality indicator may aid the medical practitioner in identifying actions and/or imaging parameters for a subsequent scan that may allow for generating the optimal view of the target structure 102. Once the optimal view is achieved, in certain embodiments, the video processor 128 may be configured to automatically position the interventional device 130, for example, to apply therapy to the target structure 102.

Embodiments of the present system 100, thus, allow for automatic transformation of an originally acquired view of the volumetric images to provide a clinically useful view of the target structure 102. Particularly, the volumetric images may be post-processed to generate the clinically useful view. In one embodiment, the post-processing may entail operations such as rotation, reorientation, clipping out irrelevant information, magnification of the target region, contrast enhancement, and/or reduction of speckle noise to provide the most optimal view of the target structure 102.

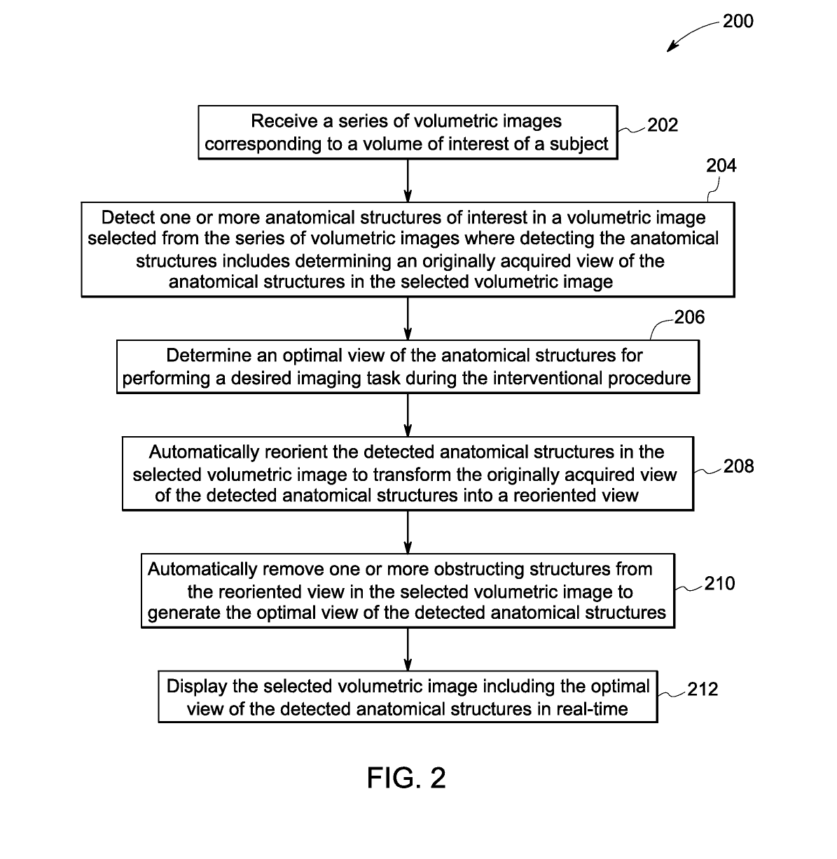

In certain embodiments, the post processing may include supplementing the originally acquired or reoriented views with the additional views acquired by the supplementary imaging system 104 to generate the optimal view for performing the desired imaging task. As previously noted, the optimal view of the target region allows for more efficient real-time guidance of the interventional device 130. An exemplary method for interventional imaging that provides an optimal visualization of a target region will be described in greater detail with reference to FIG. 2.

FIG. 2 illustrates a flow chart 200 depicting an exemplary method for imaging a subject during an interventional procedure. In the present disclosure, embodiments of the exemplary method may be described in a general context of computer executable instructions on a computing system or a processor. Generally, computer executable instructions may include routines, programs, objects, components, data structures, procedures, modules, functions, and the like that perform particular functions or implement particular abstract data types.

Additionally, embodiments of the exemplary method may also be practiced in a distributed computing environment where optimization functions are performed by remote processing devices that are linked through a wired and/or wireless communication network. In the distributed computing environment, the computer executable instructions may be located in both local and remote computer storage media, including memory storage devices.

Further, in FIG. 2, the exemplary method is illustrated as a collection of blocks in a logical flow chart, which represents operations that may be implemented in hardware, software, or combinations thereof. The various operations are depicted in the blocks to illustrate the functions that are performed, for example, during the steps of detecting one or more anatomical structures of interest, automatically reorienting the anatomical structures, and automatically removing obstructing anatomical structures. In the context of software, the blocks represent computer instructions that, when executed by one or more processing subsystems, perform the recited operations.

The order in which the exemplary method is described is not intended to be construed as a limitation, and any number of the described blocks may be combined in any order to implement the exemplary method disclosed herein, or an equivalent alternative method. Additionally, certain blocks may be deleted from the exemplary method or augmented by additional blocks with added functionality without departing from the spirit and scope of the subject matter described herein. For discussion purposes, the exemplary method will be described with reference to the elements of FIG. 1.

Interventional procedures are widely used, for example, in the management of valvular and congenital heart diseases. Increasingly, multi-modality imaging is being used during interventions for planning, guidance, and evaluation of procedure related outcomes and complications. Particularly, interventional procedures such as TEE, TTE, and ICE have been used to provide real-time, high resolution images of intracardiac anatomy and physiology. The high resolution images provide useful information for interventional device guidance. Additionally, high resolution images may also provide pathological information that may aid in providing an accurate diagnosis and/or treatment decision. For example, management of congenital heart disease and primary pulmonary hypertension may entail measurement of right ventricular volumes and function. Imaging the complex geometrical crescent shape of the right ventricle using conventional TTE or ICE procedures, however, is a challenging task. Specifically, in conventional interventional imaging systems, imaging the right ventricle may entail repeated and lengthy configuration of system controls to manually refine an FOV for imaging the right ventricle. Embodiments of the present method, however, allow for automatic adjustment of the FOV to allow for optimal visualization of anatomical structures of interest.

At step 202, where a series of volumetric images corresponding to a VOI of a subject are received. The volume of interest, for example, may correspond to biological tissues such as cardiac tissues of a patient or a non-biological material such as a stent, a plug, or a tip of a catheter. In one embodiment, the volumetric images corresponding to the VOI may be received from an imaging system such as the system 100 of FIG. 1 in real-time.

Further, at step 204, one or more anatomical structures of interest are detected in at least one volumetric image selected from the series of volumetric images. Specifically, detecting the anatomical structures entails determining an originally acquired view of the anatomical structures in the selected volumetric image. In one embodiment, the anatomical structures may be detected based on a predetermined model. For example, when imaging the pulmonary vein, one or more vessel shaped (cylindrical) models may be matched to the anatomical structures detected in the volumetric image. In another embodiment, the anatomical structures may be detected using reference shapes in a digitized anatomical atlas that fit a collection of shapes detected in the volumetric image. In one embodiment, the atlas may be generated using inputs from a clinical expert. In another embodiment, the atlas may be generated using previously acquired images of the VOI using the same or different imaging modality. Moreover, the atlas may be generated using previously acquired images of the VOI corresponding to the same subject or to a plurality of subjects corresponding to a particular demographic. Alternatively, the anatomical structures in the volumetric image may be detected using image segmentation and/or a suitable feature detection method.

In certain further embodiments, machine learning approaches may be employed to recognize features of the anatomical structures of interest such as the pulmonary vein in the volumetric image. In one example, the machine learning approaches may be employed to identify features of the anatomical structures based on high level features such as a histogram of oriented gradients (HOG). In another example, a supervised learning method may be employed, where anatomical structures of interest in a plurality of volumetric images may be manually labeled by a skilled medical practitioner. The manually labeled images may be used to build a statistical model and/or a database of true positives and true negatives corresponding to each anatomical structure of interest. In one embodiment, the manually labeled images may be used to build the model and/or database in an offline mode. However, in an alternative embodiment, the supervised learning method entails use of volumetric images that are labeled in real-time for identifying the anatomical structures of interest. The labeled volumetric images may then be used to train the supervised learning method to identify the originally acquired view of the anatomical structures in incoming volumetric images.

In certain embodiments, identifying the originally acquired view of the anatomical structures may also entail determining positions and orientations of the detected anatomical structures. In one embodiment, the positions and orientations of the anatomical structures in the originally acquired view may be determined, for example, based on segmentation or an HOG-based analysis. The determined positions and orientations of the anatomical structures in the originally acquired view may correspond to a default view of the VOI that an interventional imager such as an ICE or a TEE imaging probe is programmed to acquire. As previously noted, the originally acquired view may not be optimal for a desired imaging task. For example, an originally acquired view of the right atrium may correspond to an oblique view of the pulmonary vein that may not be suitable for ablation of desired regions of the pulmonary vein.

Accordingly, at step 206, an optimal view of one or more anatomical structures of interest for performing a desired imaging task during the interventional procedure may be determined. In one embodiment, the imaging task may include visualizing a desired view of an anatomical structure, for example, for guiding an interventional device, performing a particular interventional or diagnostic procedure, applying therapy to a desired region of an anatomical structure, adhering to a predefined imaging protocol, and/or to satisfy a user-defined input.

In certain embodiments, the optimal view may define a clinically useful spatial configuration of the anatomical structures in the volumetric image. The clinically useful spatial configuration may define a desired position and/or a desired orientation of the anatomical structures in the volumetric image that may be advantageously used to perform the desired imaging task. The optimal view including the anatomical structures in the clinically useful spatial configuration may also allow for accurate measurement of biometric parameters and/or for an efficient assessment of a pathological condition of the subject.

In certain embodiments, such an optimal view of the anatomical structures for performing the desired imaging task may be determined based on expert knowledge, standardized medical information such an a surgical atlas, a predetermined anatomical model, and/or historical information. The historical information may be derived from volumetric images and/or medical data corresponding to one or more other patients belonging to a similar demographic as the patient under investigation.

Further, at step 208, the detected anatomical structures in the selected volumetric image may be automatically reoriented to transform the originally acquired view of the detected anatomical structures into a reoriented view. In one embodiment, the reoriented view may include the detected anatomical structures in a desired spatial configuration that satisfies clinical, user-defined, and/or application-specific imaging requirements. For example, when imaging the pulmonary vein using an imaging subsystem positioned at a distal end of a catheter that is inserted into the right atrium, the originally acquired view may provide only an oblique view of the pulmonary vein. Accordingly, embodiments of the present method allow for reorientation of the pulmonary vein such that the volumetric image provides a view straight down an axis of the pulmonary vein.

In certain scenarios, reorientation alone may not provide an optimal visualization of the detected anatomical structures that may be suitable for performing the desired imaging task during the interventional procedure. For example, the reoriented view may include anatomical structures such as a septum that may occlude portions of the pulmonary vein in the reoriented view. Unlike conventional imaging systems, that entail multiple manual configurations of the system controls to clip out extraneous regions, embodiments of the present method allow for automatically removing obstructing structures from the reoriented view in the selected volumetric image, as depicted by step 210. Particularly, the obstructing structures may be removed from the reoriented view to generate an optimal view of the detected anatomical structures. As previously noted, the optimal view may correspond to a desired spatial configuration of the anatomical structures on interest that is predetermined for the desired imaging task to be performed during the interventional procedure.

Accordingly, in one example, image volume corresponding to those structures in the volumetric image that are different from the anatomical structures of interest may be analyzed. Particularly, the image volume may be analyzed to identify extraneous and/or obstructing structures in the volumetric image that occlude a view of one or more anatomical structures of interest. For example, if the analysis of the image volume indicates that an atrial septum obstructs the view of the pulmonary vein, a portion of the image volume corresponding to the septum may be automatically removed from the reoriented view. Removal of the atrial septum from the reoriented image allows for optimal visualization of the pulmonary vein, for example, for use in ablating one or more regions of the pulmonary vein with greater accuracy. Furthermore, in one embodiment, the anatomical structures in regions revealed after removal of the obstructing structures may be regenerated using previously acquired volumetric images and/or an anatomical model.

In certain embodiments, the volumetric images may also undergo additional processing for contrast enhancement, increasing a spatial resolution, and/or resizing a portion of the volumetric image to generate the optimal view. In one example, the optimal view for tracking an interventional device may entail a side view of the interventional device advancing through the patient's body to provide real-time navigational guidance during the interventional procedure. In another example, the optimal view for assessing an operation of an atrial valve includes an axial view of the valve. As previously noted, the resulting volumetric images including the optimal view may be combined with supplementary image data acquired by a supplementary imaging system to provide more comprehensive information corresponding to the target region and/or a position of the interventional devices within the patient's body.

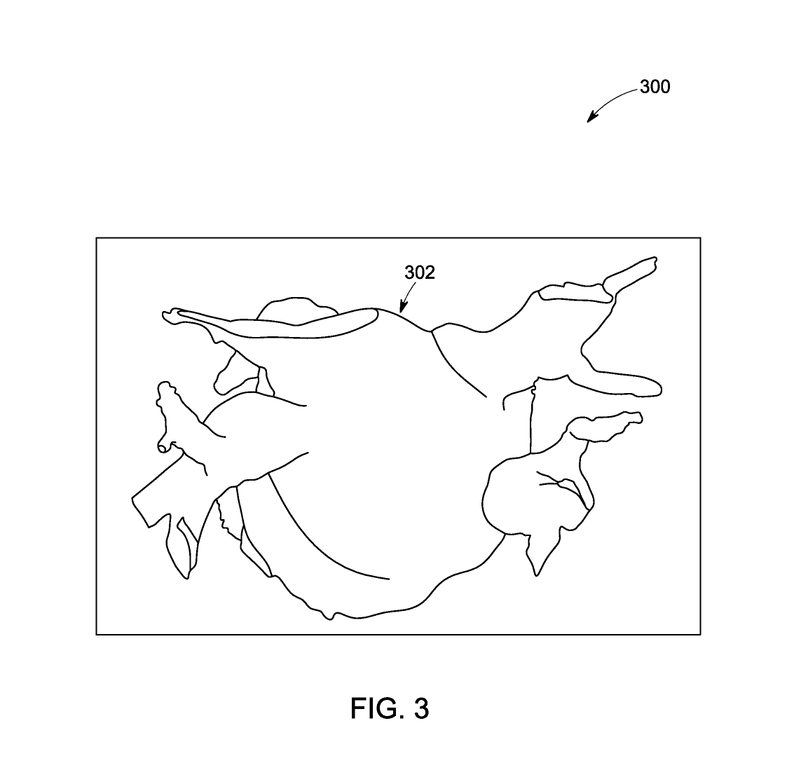

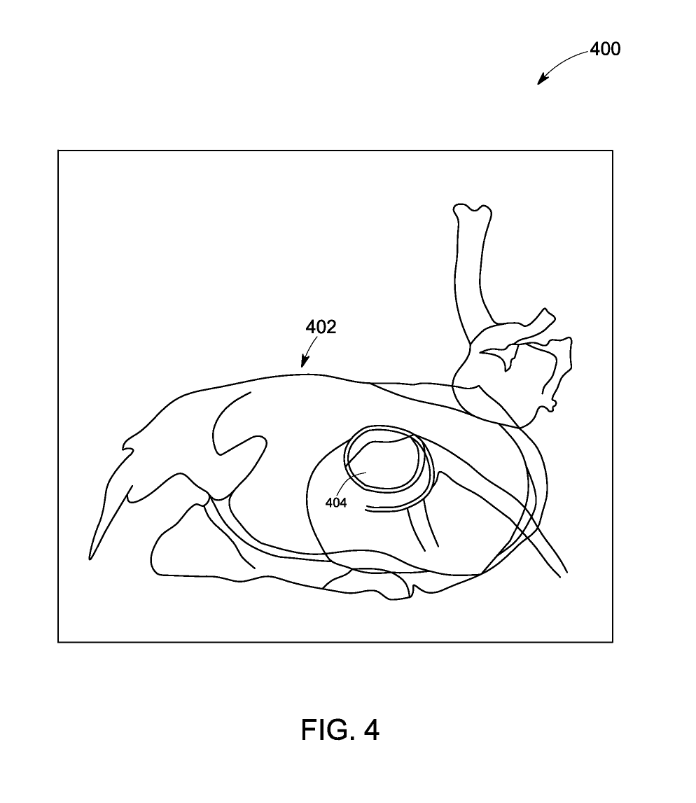

Furthermore, at step 212, the selected volumetric image including the optimal view of the detected anatomical structures may be displayed on a display device in real-time. Particularly, the optimal view may depict the repositioned, reoriented, and/or unobstructed anatomical structures in an illustrative map for providing enhanced real-time guidance of interventional devices during the interventional procedure. Additionally, the optimal view may also allow for accurate biometric measurements, which in turn, may aid in a more informed diagnosis of a medical condition of the patient. Embodiments of the present method, thus, may be used for efficient planning, guidance, and/or evaluation of progress and outcomes of the interventional procedure. Certain examples of an optimal visualization of anatomical structures using the method described with reference to FIG. 2 will be described in greater detail with reference to FIGS. 3-4.

FIG. 3 depicts a diagrammatical representation of a volumetric image 300 depicting an originally acquired view 302 of a VOI corresponding to a subject. In the embodiment depicted in FIG. 3, the VOI corresponds to a cardiac region of the subject that is imaged using a CT imaging system. Further, in one embodiment, a catheter including an imaging subsystem is navigated, for example, from the femoral artery to a right atrium of the subject to image an anatomical structure such as the pulmonary vein. As evident from the depictions of FIG. 3, the originally acquired view 302 of the VOI may not provide an optimal view of the pulmonary vein for performing the desired imaging task during an interventional procedure such as a pulmonary vein ablation. Particularly, in the originally acquired view 302, the pulmonary vein is unsuitably positioned and is occluded, thus failing to allow a medical practitioner to ablate one or more regions of the pulmonary vein with desired accuracy. The originally acquired view 302, thus, may not provide sufficient information for allowing for a real-time guidance of an ablation catheter through the patient's body during the ablation procedure. Accordingly, an embodiment of the method described with reference to FIG. 2 may be employed to process the volumetric image to provide a clinically useful visualization of the pulmonary vein.

As previously noted with reference to steps 204-206 of FIG. 2, the anatomical structures may be detected using feature detection techniques that, for example, are based on anatomical models of the cardiac region and/or supervised machine learning. Additionally, a position and orientation of each of the anatomical structures may be determined. Further, the determined position and orientation of one or more of the anatomical structures may be compared with desired positions and orientations of the anatomical structures defined by clinical protocols for the imaging task being performed. In one embodiment, the desired positions and orientations of the anatomical structures may be representative of the optimal view of the anatomical structures. The optimal view, thus generated, may provide real-time positioning and navigational guidance for interventional devices during minimally-invasive procedures.

Furthermore, the selected volumetric image may undergo one or more processing steps such as image reorientation and removal of extraneous structures to minimize or reduce a difference between the determined position and/or orientation of the anatomical structures and the desired position and/or orientation of the of the anatomical structures defined in the optimal view. Certain examples of automated post-processing the volumetric images to generate an optimal view of the anatomical structures and/or to minimize the difference between the determined position and/or orientation and the desired position and/or orientation of the anatomical structures were previously described with reference to FIG. 2. An example of an optimal view of the anatomical structures generated using the method of FIG. 2 may be depicted in FIG. 4.

FIG. 4 is a diagrammatical representation of a volumetric image 400 including an optimal view 402 that is representative of a reoriented and/or repositioned VOI of FIG. 3. In one example, the reoriented view is generated using the method of FIG. 2. Particularly, the spatial configurations of the anatomical structures depicted in FIG. 3 are automatically reoriented to visualize the rim of a pulmonary vein 404 in the center of the volumetric image 400.

Additionally, the image 300 (see FIG. 3) may be uncluttered by clipping out extraneous and/or obstructive regions. Reorientation and/or uncluttering of the image 300 transforms the originally acquired view 302 of the anatomical structures depicted in FIG. 3 to the optimal view 402 in FIG. 4 that provides better guidance for pulmonary vein ablation. In one embodiment, the optimal view 402 may aid in providing real-time guidance to the medical practitioner to accurately position and/or move an ablation catheter within the cardiac region of the patient. Specifically, the optimal view 402 that depicts a straight view down an axis of the cylindrical shape of the pulmonary vein 404 may allow the medical practitioner to trace the rim of the pulmonary vein 404 to ablate target regions, while keeping track of previously ablated regions along the rim of the pulmonary vein 404.

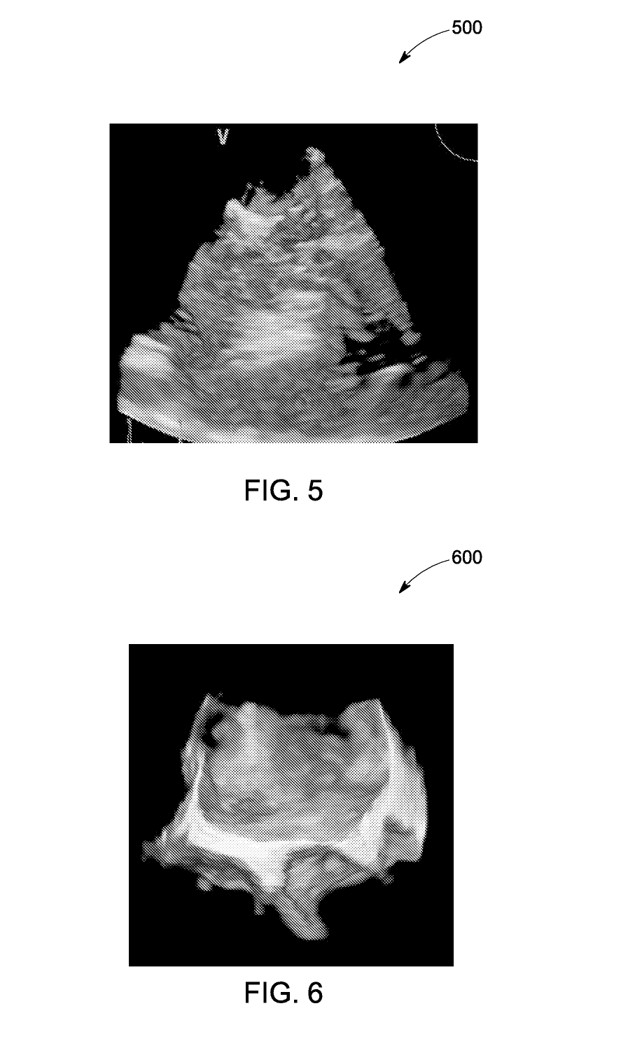

Moreover, FIG. 5 depicts an exemplary volumetric image 500 of a default side view of a cardiac valve acquired by a TEE probe. The originally acquired side view depicts an image volume corresponding to the cardiac valve that is generated using a predefined FOV employed by the TEE probe. Similarly, FIG. 6 depicts an exemplary volumetric image 600 of a default axial view of the cardiac valve of FIG. 5 that is acquired using another predefined FOV employed by the TEE probe. In the example depicted in FIG. 6, the default axial view corresponds to a predefined view that is predominantly aligned with a direction of ultrasound signals used to image the valve.

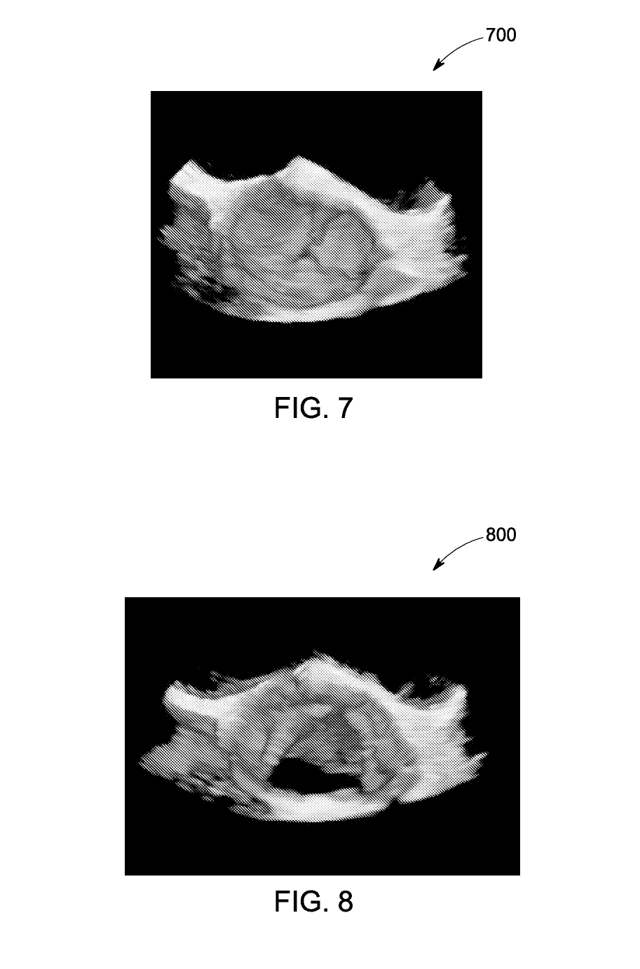

Further, FIG. 7 illustrates an exemplary image 700 depicting an optimal view of the cardiac valve of FIG. 5. In one embodiment, the optimal side view is generated using the method described with reference to FIG. 2. Specifically, the optimal side view is generated by cropping the volumetric image 500 of FIG. 5 to remove extraneous and/or obstructing structures. Additionally, the anatomical structures in the volumetric image 500 are reoriented to generate the image 700 that visualizes regions straight down the axis of the valve. Specifically, the image 700 depicts the optimal side view of the valve, when the valve is closed. Such an optimal side view of the valve may be used to provide guidance for coaxial alignment of an interventional probe during valve replacement and repairs. Furthermore, the optimal view of the closed valve allows an assessment of valve operation. For example, the optimal side view of the closed valve may allow the medical practitioner to determine if there is a leak in the valve, and a cause of the leak based on an extent of closure of the valve during different cardiac cycles.

Similarly, FIG. 8 illustrates an exemplary image 800 depicting an optimal view of the cardiac valve of FIG. 5, when the valve is open. Specifically, the image 800 depicts the optimal axial view of the valve generated using the method described with reference to FIG. 2. In one example, the optimal axial view of the valve may be used to provide guidance for centering the interventional probe during valve replacement and repair procedures. Furthermore, as previously noted, the optimal axial view of the open valve may also allow for an assessment of valve operation, such as to determine a presence and cause of a leak in the valve due to improper closure of the valve. Automatically optimizing the visualization of the valve through automated reorienting and uncluttering of the originally acquired views provides clinically useful information, while allowing substantial savings in imaging time that are typically not available with conventional interventional imaging systems.

Although embodiments of the present methods and systems disclose optimal visualization of a cardiac valve and a pulmonary vein for use during an ablation procedure, in alternative embodiments, the present methods and systems may also be used in other interventional procedures. For example, embodiments of the present methods and systems may be used in interventional procedure corresponding to left atrial appendage closures, patent foramen ovale closures, atrial septal defects, mitral valve repair, aortic valve replacement, and/or CRT lead placement.

Embodiments of the present system and methods, thus, allow for optimal visualization of anatomical structures in a VOI. Particularly, embodiments described herein allow for determining a desired view for desired imaging tasks. The desired view defines a spatial position and/or orientation of the anatomical structures that may be most suitable for performing the desired imaging tasks such as biometric measurements and/or analysis. Accordingly, each of the volumetric images may be adapted to substantially match the desired view. Such an automatic view control provided by embodiments of the present systems and methods results in a substantial reduction in imaging time, which in turn, reduces a rate of complications and/or a need for additional supplementary procedures.

It may be noted that the foregoing examples, demonstrations, and process steps that may be performed by certain components of the present systems, for example by the processing unit 120 and/or the video processor 128 of FIG. 1, may be implemented by suitable code on a processor-based system. To that end, the processor-based system, for example, may include a general-purpose or a special-purpose computer. It may also be noted that different implementations of the present disclosure may perform some or all of the steps described herein in different orders or substantially concurrently.

Additionally, the functions may be implemented in a variety of programming languages, including but not limited to Ruby, Hypertext Preprocessor (PHP), Perl, Delphi, Python, C, C++, or Java. Such code may be stored or adapted for storage on one or more tangible, machine-readable media, such as on data repository chips, local or remote hard disks, optical disks (that is, CDs or DVDs), solid-state drives, or other media, which may be accessed by the processor-based system to execute the stored code.

Although specific features of embodiments of the present disclosure may be shown in and/or described with respect to some drawings and not in others, this is for convenience only. It is to be understood that the described features, structures, and/or characteristics may be combined and/or used interchangeably in any suitable manner in the various embodiments, for example, to construct additional assemblies and methods for use in diagnostic imaging.

1. A method for imaging a subject, comprising:
receiving a series of volumetric images corresponding to a volume of interest in the subject during an interventional procedure;
detecting one or more anatomical structures in at least one volumetric image selected from the series of volumetric images, wherein detecting the anatomical structures comprises determining an originally acquired view of the anatomical structures in the selected volumetric image;
determining an optimal view of the one or more anatomical structures of interest for performing a desired imaging task during the interventional procedure;
automatically reorienting the detected anatomical structures in the selected volumetric image to transform the originally acquired view of the detected anatomical structures into a reoriented view;
automatically removing one or more obstructing structures from the reoriented view in the selected volumetric image to generate the optimal view of the detected anatomical structures; and
displaying the selected volumetric image comprising the optimal view of the detected anatomical structures in real-time.

2. The method of claim 1, wherein the series of volumetric images comprises a plurality of time varying three-dimensional image volumes corresponding to the subject.

3. The method of claim 1, wherein determining the optimal view for performing the desired imaging task comprises identifying the optimal view based on expert knowledge, a predetermined model corresponding to the volume of interest, a machine learning method, user input, or combinations thereof.

4. The method of claim 1, wherein detecting the one or more of the anatomical structures in the selected volumetric image comprises identifying the anatomical structures based on expert knowledge, a predetermined model corresponding to the volume of interest, a machine learning method, user input, or combinations thereof.

5. The method of claim 1, wherein automatically reorienting the detected anatomical structures comprises reducing a difference between a determined orientation of the detected anatomical structures in the originally acquired view and a desired orientation of the anatomical structures defined by the optimal view.

6. The method of claim 1, wherein automatically reorienting the detected anatomical structures comprises reducing a difference between a determined position of the anatomical structures in the originally acquired view and a desired position of the anatomical structures defined by the optimal view.

7. The method of claim 1, wherein automatically removing one or more obstructing structures comprises removing extraneous portions of the volumetric image based on the desired imaging task to be performed during the interventional procedure.

8. The method of claim 7, wherein removing extraneous portions comprises one or more of clipping, cropping, and segmenting the selected volumetric image.

9. The method of claim 1, further comprising performing a contrast enhancement, increasing a spatial resolution, resizing a portion of the volumetric image, or combinations thereof, to generate the optimal view of the detected anatomical structures in the selected volumetric image.

10. The method of claim 1, further comprising providing real-time guidance for navigating an interventional device in real-time using the selected volumetric image comprising the optimal view.

11. An imaging system, comprising:
an acquisition subsystem configured to acquire a series of volumetric images corresponding to a volume of interest in a subject;
a processing unit communicatively coupled to the acquisition subsystem and configured to:
detect one or more anatomical structures in at least one volumetric image selected from the series of volumetric images, wherein detecting the anatomical structures comprises determining an originally acquired view of the anatomical structures in the selected volumetric image;
determine an optimal view of the one or more anatomical structures of interest for performing a desired imaging task during the interventional procedure;
automatically reorient the detected anatomical structures in the selected volumetric image to transform the originally acquired view of the detected anatomical structures into a reoriented view;
automatically remove one or more obstructing structures from the reoriented view in the selected volumetric image to generate the optimal view of the detected anatomical structures; and
a display operatively coupled to at least the processing unit and configured to display the selected volumetric image comprising the optimal view of the detected anatomical structures in real-time.

12. The system of claim 11, wherein the acquisition subsystem comprises an ultrasound system, a magnetic resonance imaging system, a computed tomography system, a positron emission tomography system, an optical coherence tomography system, an electrophysiology system, an X-ray system, an interventional imaging system, or combinations thereof.

13. The system of claim 12, further comprising a supplementary imaging system, wherein the supplementary imaging system comprises an ultrasound system, a magnetic resonance imaging system, a computed tomography system, a positron emission tomography system, an optical coherence tomography system, an electrophysiology system, an X-ray system, an interventional imaging system, or combinations thereof.

14. The system of claim 13, wherein the processing unit is configured to:
receive supplementary information corresponding to the volume of interest from the supplementary imaging system; and
detect the anatomical structures, identify the obstructing structures, determine the optimal view, determine the reoriented view, or combinations thereof, based on the supplementary information.

15. A non-transitory computer readable medium that stores instructions executable by one or more processors to perform a method for imaging a subject, comprising:
detecting one or more anatomical structures in at least one volumetric image selected from the series of volumetric images, wherein detecting the anatomical structures comprises determining an originally acquired view of the anatomical structures in the selected volumetric image;
determining an optimal view of one or more anatomical structures of interest for performing a desired imaging task during the interventional procedure;
automatically reorienting the detected anatomical structures in the selected volumetric image to transform the originally acquired view of the detected anatomical structures into a reoriented view;
automatically removing one or more obstructing structures from the reoriented view in the selected volumetric image to generate the optimal view of the detected anatomical structures; and
displaying the selected volumetric image comprising the optimal view of the detected anatomical structures in real-time.
